(19) United States
(12) Patent Application Publication
(10) Pub. No.: US 2023/0372555 A1
(43) Pub. Date: Nov. 23, 2023

(54) PHOTO REACTOR DEVICES, SYSTEMS, AND METHODS OF USE THEREOF

(71) Applicant: Colorado State University Research Foundation, Fort Collins, CO (US)

(72) Inventors: Bryan D. Willson, Fort Collins, CO (US); Raymond P. Goodrich, Fort Collins, CO (US); John C. Mizia, Fort Collins, CO (US); Matthew R. Willman, Fort Collins, CO (US)

(21) Appl. No.: 17/913,528

(22) PCT Filed: Mar. 24, 2021
(86) PCT No.: PCT/US21/23996

§ 371 Date: Sep. 22, 2022

Related U.S. Application Data
(60) Provisional application No. 62/994,258, filed on Mar. 24, 2020.

(51) Int. Cl. A61L 2/00; A61L 2/10

(52) U.S. Cl. CPC A61L 2/0047; A61L 2/10; A61L 2202/22

(57) ABSTRACT

A device for emitting photons into a fluid is disclosed. The device includes a fluid channel configured to transfer the fluid from an inlet to an outlet thereof and the fluid channel having a helical pathway wound about a longitudinal axis. The device also includes at least one inner light source adjacent the fluid channel, the at least one inner light source positioned between the fluid channel and the longitudinal axis and at least one outer light source adjacent the fluid channel, the fluid channel positioned between the outer light source and the longitudinal axis.

### CROSS REFERENCE TO RELATED APPLICATION

This application claims priority from U.S. provisional patent application No. 62/994,258, titled “Methods, Apparatus, and Compositions for Inactivating Viral Particles and Other Pathogens,” filed on Mar. 24, 2020, the entirety of which is incorporated by reference herein.

### FIELD OF THE INVENTION

This application relates to compositions, devices, systems, and methods for emitting and directing photons into a flowing fluid. In particular, this application relates to devices, systems, and methods for inactivating viral particles and other pathogens.

### BACKGROUND OF THE INVENTION

Some current pathogen reduction or viral deactivation technologies involve substances and/or processes that are harmful to biologic materials. Using such harmful substances and processes typically requires removal of any residual substance after viral deactivation, which presents a risk of incomplete removal of the harmful substance and additional time and expense for subsequent processing. Thus, a need exists to provide safer and more efficient viral inactivation compositions, devices, systems, and methods.

### SUMMARY OF THE INVENTION

In a first exemplary embodiment of the present invention, a device for emitting photons into a fluid may include a fluid channel configured to transfer the fluid from an inlet to an outlet thereof and the fluid channel having a helical pathway wound about a longitudinal axis. The device may also include at least one inner light source adjacent the fluid channel, the at least one inner light source positioned between the fluid channel and the longitudinal axis and at least one outer light source adjacent the fluid channel, the fluid channel positioned between the outer light source and the longitudinal axis.

In some versions of the first embodiment, at least one of the inner and outer light sources may be mounted at a top surface and extend downwardly parallel with the longitudinal axis and at least a second one of the inner and outer light sources may be mounted at a bottom surface axially spaced apart from the top surface and extend upwardly parallel with the longitudinal axis. The at least one outer light source may include a plurality of light sources arranged circumferentially around the helical pathway. Also, at least one of the inner and outer light sources may be a fluorescent light source. At least a second one of the inner and outer light sources may be an LED light source. Additionally, at least one of the inner and outer light sources may be a narrowband wavelength light source. At least two of the inner and outer light sources may have different peak wavelengths.

In a second exemplary embodiment of the present invention, a device for emitting photons into a fluid may include a fluid channel configured to transfer the fluid from an inlet to an outlet thereof and at least one first light source and at least one second light source adjacent the fluid channel. The at least one first light source may have a different peak wavelength than the at least one second light source and the at least one first light source may be configured to have a narrowband wavelength output. Also, at least one of the first and second light sources may be a fluorescent light source.

In some versions of the second exemplary embodiment, at least one of the first and second light sources may be an LED light source. The at least one first light source and the at least one second light source may be florescent light sources. Also, at least one of the first and second light sources may have a peak UV-B wavelength. At least one of the first and second light sources may have a peak UV-C wavelength. In addition, at least one of the first and second light sources may have a peak UV-C wavelength. At least one of the first and second light sources may have a peak wavelength outside of UV-B and UV-C.

In a third exemplary embodiment of the present invention, a method of directing photons into fluid along a pathway between an inlet and an outlet may include emitting, with at least one first light source, photons having a narrowband first peak wavelength into the fluid pathway and emitting, with at least one second light source, photons having a second peak wavelength different than the first wavelength into the fluid pathway. At least one of the first and second light sources may be a fluorescent light source.

In some versions of the third exemplary embodiment, at least one of the first and second light sources may be an LED light source. The at least one first light source and the at least one second light source may be florescent light sources. Also, at least one of the first and second light sources may have a peak UV-B wavelength. At least one of the first and second light sources may have a peak UV-C wavelength. Further, at least one of the first and second light sources may have a peak UV-C wavelength. At least one of the first and second light sources may have a peak wavelength outside of UV-B and UV-C.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of a photo reactor according to an exemplary embodiment of the present disclosure.

FIG. 2 is a perspective view of some of the components of the photo reactor from FIG. 1.

FIG. 3 is a perspective view of a lamp subassembly from FIG. 1.

FIG. 4 is a is a top sectional view of the lamp subassembly of FIG. 3 taken along section line 4-4 in FIG. 3.

FIG. 5 is a perspective longitudinal sectional view of the photo reactor of FIG. 2 taken along section line 5-5 in FIG. 2.

FIG. 6 is a perspective view of some of the components of the photo reactor from FIG. 1.

FIG. 7 is a top view of a coil assembly according to an exemplary embodiment of the present disclosure.

FIG. 8 is a perspective longitudinal sectional view of the coil subassembly from FIG. 7 as taken along section line 8-8 in FIG. 7.

FIG. 9 is a perspective longitudinal sectional view of a component of the coil subassembly from FIG. 7 as taken along section line 8-8 in FIG. 7.

FIG. 10 is a perspective longitudinal sectional view of a component of the coil assembly from FIG. 7 as taken along section line 8-8 in FIG. 7.

FIG. 11 is a is a perspective view of a lamp subassembly according to an exemplary embodiment of the present disclosure.

FIG. 12 is a is a top sectional view of the lamp subassembly of FIG. 11.

FIG. 13 is a is a perspective view of a lamp subassembly according to an exemplary embodiment of the present disclosure.

FIG. 14 is a is a top sectional view of the lamp subassembly of FIG. 13.

FIG. 15 is a is a perspective view of a lamp subassembly according to an exemplary embodiment of the present disclosure.

FIG. 16 is a is a top sectional view of the lamp subassembly of FIG. 15.

FIG. 17 is a is a perspective view of a lamp subassembly according to an exemplary embodiment of the present disclosure.

FIG. 18 is a is a top sectional view of the lamp subassembly of FIG. 17.

### DETAILED DESCRIPTION

Reference will now be made in detail to the exemplary embodiments, which are illustrated in the accompanying drawings. Wherever possible, the same reference numbers will be used throughout the drawings to refer to the same or like parts.

Described herein are compositions, devices, systems, and methods for inactivating viruses, including the COVID-19 virus. The compositions, devices, systems, and method disclosed herein take advantage of a unique property of a photosensitizer riboflavin and UV light to selectively inactivate virus particles by directed damage to nucleic acids while preserving the integrity of the proteins and other viral antigens. The nature of the photosensitizer (riboflavin) may provide for low toxicity and thus easy handling, distribution, and processing under even austere conditions.

The compositions, devices, systems, and methods described herein, can be used to disinfect, e.g., inactivate, a wide variety of substances (liquid, solid, etc.), surfaces, equipment, etc. that may be or are contaminated with viral particles and/or other pathogens.

One advantage of using some of the methods disclosed herein is that the photosensitizer, riboflavin, is inexpensive, has been demonstrated to be non-toxic and may not pose safety or environmental concerns. In the processes described herein, the virus or target agent is inactivated in situ in its native form. The processes described herein inactivate nucleic acid replication without requiring additional processing steps to remove replication potential in the target agent.

In some embodiments, the inactivated viral particles are adenovirus particles, adeno-associated virus (AAV) particles, lentivirus particles, coronavirus particles or retrovirus particles. In some embodiments, the inactivated viral particles are inactivated COVID-19 virus particles. In some embodiments, the viral particles are chikungunya particles, or MERS-coV particles. In some embodiments, the inactivated viral particles are Dengue, Zika, Influenza (e.g., A, B, C), Marburg, Rabies, Human Immunodeficiency Virus (HIV), Smallpox, Hantavirus, Rotavirus, SARS-CoV, MERS-CoV, Cytomegalovirus (CMV), Ebola, Epstein-Barr, Herpes (e.g., 1, 2, 6, 7, 8), Hepatitis (e.g., A, B, C, D, E), Human Papillomavirus, Mumps, Measles, Rubella, Polio, Varicella Zoster, Respiratory Syncytial Virus (RSV), Semliki Forest, West Nile, Yellow Fever, or Vesicular Stomatitis particles.

The inactivated viral particles described herein may be produced using an innocuous chemical agent in a selective process that prevents cellular replication. More specifically, the inactivated viral particles may be produced by the combined application of a photosensitizer and light for rendering viral particles unable to cause disease. The process for producing the inactivated viral vaccines of the disclosure is described in detail below.

Initially, a surface or substance may be provided which may be or is contaminated with viral particles. Next, the viral particles are inactivated using photochemical technology. This is achieved using photosensitizers that can act as electron transfer agents. The application of photosensitizer agents that can be placed into an excited state in proximity to a guanine base in DNA or RNA constructs may allow for selective modification (e.g. oxidation, cross-linking, fragmentation, deamination) of these bases. Because electron chemistry can only occur over short distances, the photosensitizer agent must be bound or associated with (i.e., intercalated with) the nucleic acid in order to carry out the desired chemistry.

In some embodiments, the photosensitizer is a flavin, for example riboflavin (Vitamin B2), flavin mononucleotide, or flavin adenine dinucleotide. In some embodiments, the photosensitizer is a tertiary aliphatic amine (e.g., 1,4-diazabicyclo(2,2,2)octane), a piperazine, (e.g., N-2-hydroxyethylpiperazine-N′-2-ethanesulfonic acid and 1,4-dimethylpiperazine), an amino acid (e.g., tyrosine, tryptophan, histidine, methionine), an enzyme (e.g., superoxide dismutase) or EDTA (ethylenediaminetetraacetic acid). In some embodiments, the photosensitizer is riboflavin.

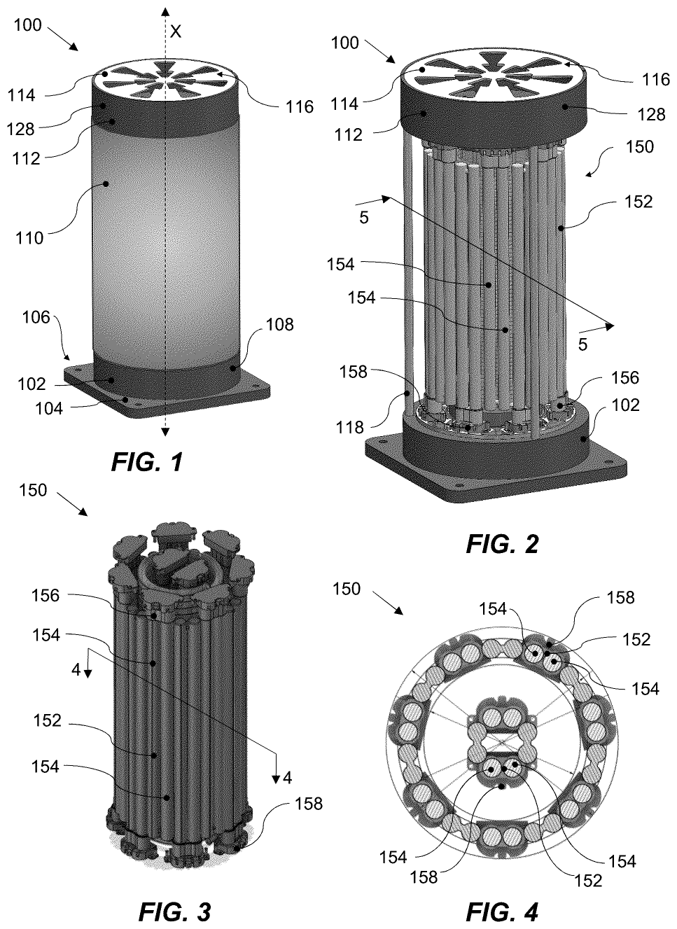

FIGS. 1-6 depict an exemplary embodiment of a photo reactor 100 for emitting and directing photons into a flowing fluid, such as a solution containing photosensitizer, such as riboflavin. In some embodiments, the concentration of photosensitizer used during inactivation is about 10 μM to about 100 μM, such as about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

In some embodiments, the solution contains the photosensitizer at a concentration of about 1 μM to about 50 μM, such as about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, or about 50 μM. In some embodiments, the photosensitizer concentration is less than about 10 μM, such as less than about 9 μM, about 8 μM, about 7 μM, about 6 μM, about 5 μM, about 4 μM, about 3 μM, about 2 μM, or about 1 μM.

The photo reactor 100 may be included in a photo reactor system which may include a pump (not shown) such as a positive displacement pump for pumping the fluid and at least one reservoir (not shown) for storing the fluid. With reference to FIG. 1, the photo reactor 100 includes a base 102 which may include a substantially flat flange portion 104 for resting on a flat surface. The flange portion 104 may define one or more apertures 106 for receiving a corresponding fastener for securing the photo reactor 100 to the flat surface. The base 102 may also include a cylindrical portion 108 extending upward from the flange portion 104 along a longitudinal axis X. The cylindrical portion 108 may include a plurality of radial apertures (not shown) for providing a passageway therethrough for electrical cables, tubing, and/or ventilation air. A reflective sleeve 110 may extend axially between the cylindrical portion 108 of the base 102 and a cylindrical portion 128 of a top cap 112 located at an end opposite from the base 102. The reflective sleeve 110 may have a cylindrical shape with a mirrored inner surface for reflecting light inwardly. The mirrored inner surface may comprise an oxidized coating such as ZnO₂, Y₂O₃, ThO₂, Sc₂O₃, MgO, Al₂O₃, HfO₂, TiO₂, SiO₂ or various combinations thereof. The top cap 112 may have a cylindrical shape with a vent plate 114 at an axially most distal end. The vent plate 114 may have or define one or more axial apertures 116 for providing a passageway therethrough for electrical cables, tubing, and/or ventilation air.

FIG. 2 shows the photo reactor 100 with the reflective shield 110 removed. One or more support rods 118 may extend axially between the cylindrical portion 108 of the base 102 and the top cap 112. The support rods 118 may support the top cap 112 by itself or in conjunction with the reflective shield 110. In some embodiments the support rods 118 may be removed and the top cap 112 may be supported only by the reflective shield 110. The cylindrical portion 108, the top cap 112, and the reflective shield 110 may house a lamp subassembly 150. The lamp subassembly 150 includes one or more lamps 152, with each lamp 152 having a base 156 for connecting one or more bulbs 154 arranged parallel to the longitudinal axis X.

As best shown in FIGS. 3 and 4, the lamp subassembly 150 has eighteen lamps 152 with each lamp 152 having two bulbs 154 (each lamp has two halves, but it is only one lamp). The base 156 may include a receptacle for receiving one or more bulbs 154 and at least two pins opposite the receptacle for making an electrical connection in a corresponding socket 158. The base 156 may be a 2G11-type base, which has four pins. It is envisioned that the lamp assembly 150 may include other lamp and/or bulb types or geometries known to those having ordinary skill in the art. The lamps 152 of the subassembly 150 may be arranged along two concentric circles and may be alternatingly mounted to the base 102 and the top cap 112. For example, as shown in FIG. 3, seven lamps 152 and sockets 158 may be arranged in a heptagon orientation extending downwardly circumscribing two inner lamps 152 that also extend downwardly. Between each pair of bulbs 154 of adjacent downwardly extending lamps 152 is a pair of upwardly extending bulbs 154. The upwardly extending lamps 152 include seven corresponding lamps 152 also arranged in the shape of a heptagon, but out of phase by 360 degrees/number of lamps 152, which is approximately 51 degrees from the seven downwardly extending lamps 152, and two corresponding inner lamps 152 that also extend upwardly, but out of phase by 90 degrees from the two downwardly extending lamps 152. Other lamp subassembly 150 arrangements have been contemplated and are discussed in more detail below.

Returning to the bulbs 154, the lamp subassembly 150 may include fluorescent bulbs configured to emit visible, ultra-violate (UV), and/or infrared light within broad or narrow bandwidths. For example, the bulbs 154 may be fluorescent bulbs configured to emit a wide bandwidth of UV-A and UV-B wavelengths, such as a bandwidth between approximately 275 nm and 375 nm. The bulbs 154 may be germicidal fluorescent bulbs configured to emit a narrow band of UV-C wavelength, such as a narrow bandwidth centered around 253.7 nm. The bulbs 154 may also include LED bulbs with narrow bandwidths, although wide bandwidths are also possible. As some non-limiting examples, the LED bulbs may be configured to have an approximately 10 nm bandwidth or smaller centered at a peak wavelength of 265 nm, 275 nm, 310 nm, 365 nm, 395 nm, or 405 nm. A peak wavelength may be the largest amplitude of a wavelength emitted from the entire spectrum of light emitted from the light source or it may be the wavelength associated with largest amount of energy emitted for a narrow bandwidth at each localized amplitude peak emitted from the light source. The lamps 152 of the lamp subassembly 150 may be configured to have any broadband or narrowband fluorescent or LED bulb or combination thereof. For example, the lamp subassembly 150 may be comprised of 18 fluorescent bulbs configured to emit wide bandwidth UV-A and UV-B wavelengths and 18 LED bulbs configured to emit 265 nm wavelength light. The lamp subassembly 150 may be configured for quick replacement of any number of the bulbs 154 as necessary without removing the entire lamp subassembly 150. In addition, as will be described in more detail below, the lamps 152 and/or the bulbs 154 may be selectively enabled or disabled for various reasons. Moreover, some of the bulbs may be pulsed on and off to have a desired duty cycle, particularly for LED bulbs. Further, LED bulbs having different wavelengths may be cycled on and off. The following is an example of how the photo reactor 100 may be configured and reconfigured. The photo reactor 100 may have a lamp subassembly 150 comprising 36 fluorescent bulbs configured to emit wide bandwidth UV-A and UV-B wavelengths. An operator may pass a quantity of a fluid through the photo reactor 100. Next, the operator may electronically switch off 18 of the bulbs 152 for reducing power and heat generation and then pass another quantity of a fluid through the photo reactor 100. Next, the operator may reconfigure the lamp subassembly 150 to have 18 fluorescent bulbs configured to emit wide bandwidth UV-A and UV-B wavelengths and 18 LED bulbs configured to emit 265 nm wavelength light and switch back on the 18 bulbs which were previously turned off. The operator may then pass another quantity of a fluid through the photo reactor 100. Next, the operator may then turn off the 18 fluorescent bulbs and pass another quantity of a fluid through the photo reactor 100. Finally, the operator may then turn on the 18 fluorescent bulbs and turn off the 18 LED bulbs and pass another quantity of a fluid through the photo reactor 100. Although the examples above discuss fluorescent and LED bulbs configured to emit UV wavelengths of light, the lamp subassembly may comprise fluorescent and/or LED bulbs configured to emit visible or infrared wavelengths of light. In addition, the lamp subassembly 150 may also be configured to use incandescent bulbs, halogen lamps, arc lamps, and gas-discharge lamps.

In some embodiments, the UV light may have a wavelength of 170 to 400 nm, including all ranges and subranges therebetween. For example, in some embodiments, the UV light has a wavelength of 315 to 400 nm, 310 to 320 nm, 280 to 360 nm, 280 to 315 nm, or 180 to 280 nm. In some embodiments, viral particles may be treated with multiple wavelengths of light simultaneously. In some embodiments where riboflavin is used as a photosensitizer, UV light having a wavelength of 310 to 320 nm may be used. The inventors have determined that this wavelength prevents riboflavin from reacting in free solution, which results in production of undesirable oxygen free radicals. At these wavelengths, riboflavin may selectively react when intercalated with nucleic acid.

The dose of the UV light may vary depending on the volume of solution being treated. For example, the dose of the UV light may be between 200-400 Joules (e.g., 300 Joules) for a volume of about 170 to 370 ml of solution. As will be understood by those of skill in the art, the dosage may be adjusted up or down if the volume to be treated is above or below this range.

In some embodiments, the dose of UV light may be from about 200 Joules to about 600 Joules, for example about 200, about 225, about 250, about 275, about 300, about 325, about 350, about 375, about 400, about 425, about 450, about 475, about 500, about 525, about 550, about 575, or about 600 Joules. In some embodiments, the volume of viral preparations for illumination may be from about 200 ml to about 600 ml, for example about 200, about 225, about 250, about 275, about 300, about 325, about 350, about 375, about 400, about 425, about 450, about 475, about 500, about 525, about 550, about 575, or about 600 ml. In some embodiments, the dose of UV light may be from about 0.5 Joules/ml to about 3.0 Joules/ml. For example, the dose of UV light may be about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 Joules/ml.

In some embodiments, the light treatment comprises treatment with light from a blue LED. In some embodiments, the wavelength of the light is 300 nm to 500 nm. In some embodiments, the wavelength is about 450 nm. In an embodiment, the wavelength is about 447 nm.

As discussed above, the total energy or per unit volume can adjusted. This may be done by adjusting the pump speed, selecting a tubing 132 having a given length/diameter, and/or activating and deactivating various lamps 152. Thus depending on the desired total energy or energy per unit volume, the viral particles may be treated with light for about 1 minute to about 60 minutes, for example, about 1, about 2, about 3, about 4, about 5, about 6, about 7, about 8, about 9, about 10, about 15, about 20, about 25, about 30, about 35, about 40, about 45, about 50, about 55, or about 60 minutes. In some embodiments, the viral particles are treated with UV light for about 1 minute to about 10 minutes, about 1 minute to about 5 minutes, or about 1 minute to about 3 minutes.

In some embodiments, the substance/surface/etc. containing viral particles is preincubated/pretreated for a predetermined period of time in the solution containing the photosensitizer (e.g., riboflavin) before subjecting the viral particles to the light treatment.

In some embodiments, the viral particles are not subjected to any additional inactivation steps after light treatment.

In some embodiments, a solution containing the photosensitizer (e.g., riboflavin) may contain other components. Some examples include: detergents, surfactants, solvents, alcohols, and/or combinations thereof.

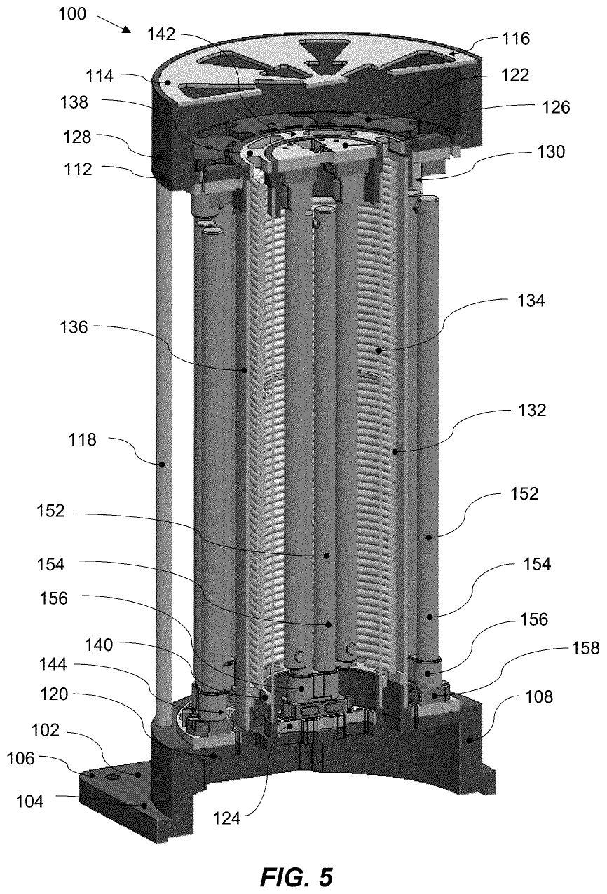

Returning to the figures, FIG. 5 is a perspective longitudinal sectional view of the photo reactor 100 showing internal features thereof. The base 102 may include an annular base mounting surface 120 extending radially inward from the cylindrical portion 108 for mounting the socket 158 of each upwardly extending lamp 152 from the outer circumferentially arranged lamps 152. Likewise, the top cap 112 may include an annular top cap mounting surface 122 extending radially inward from the cylindrical portion 128 for mounting the socket 158 of each downwardly extending lamp 152 from the outer circumferentially arranged lamps 152. Both of the base and top cap annular mounting surfaces 120, 122 may include a plurality of vent holes extending therethrough. The base 102 may also include a lower central mounting surface 124 removably attachable to the base 102 and positioned radially inwardly and centrally with respect to the base mounting surface 120. The sockets 158 of the upwardly extending inner lamps 152 may be mounted on the lower central mounting surface 124. Similarly, the top cap 112 may also include an upper central mounting surface 126 removably attachable to the top cap 112 and positioned radially inwardly and centrally with respect to the top cap mounting surface 122. The sockets 158 of the downwardly extending inner lamps 152 may be mounted on the upper central mounting surface 126.

The photo reactor 100 may also include a coil subassembly 130 positioned between the inner and outer lamps 152 of the lamp subassembly 150. The coil subassembly 130 may include an inner cylindrical shield 134 positioned adjacent and radially outward with respect to the inner lamps 152. The coil subassembly 130 may include an outer shield 136 spaced apart from the inner shield 134 and positioned adjacent and radially inward with respect to the outer lamps 152. The inner and outer shields 134, 136 may comprise a rigid material translucent to UV wavelengths, such as quartz. To help keep the spacing between the inner and outer shields 134, 136, an annular top vent plate 138 and an annular bottom vent plate 140 may be positioned between the inner and outer shields at respective top and bottom ends thereof. The top vent plate 138 may define one or more apertures 142 for providing a passageway therethrough for tubing and/or ventilation air. Similarly, the bottom vent plate 140 may define one or more apertures 144 for providing a passageway therethrough for tubing and/or ventilation air. At least one of the top or bottom vent plates 138, 140 may be removable for installing tubing 132 into and removing tubing 132 from the space between the inner and outer shields 134, 136.

The coil subassembly 130 may also include tubing 132 helically wound within the spacing between the inner and outer shields 134, 136 and having ends that may extend through the apertures 142, 144 of the top and bottom vent plates 138, 140, respectively. The tubing 132 may be Class VI tubing and comprised of a material at least partially translucent to UV light, such as FEP or PTFE. The inner and outer shields 134, 136 may provide structural support for the tubing 132 and may also help insulate fluid passing through the tubing 132 from heat not directly radiated by the bulbs 154 into the fluid.

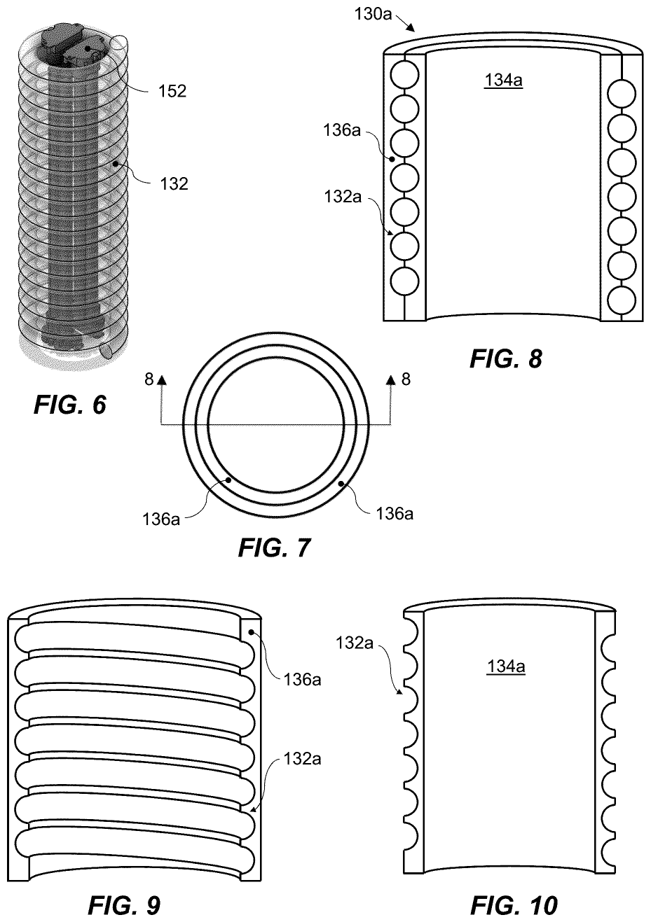

The space between the inner and outer shields 134, 136 may be configured to accommodate tubing of different diameters. For example, an operator may use a smaller diameter tubing 132, such as ¼ inch outer diameter tubing shown in FIG. 5, for a fluid that requires more extensive bombardment of photons, whereas FIG. 6 shows the inner bulbs 152 surrounded by tubing 132 having a larger diameter, such as ⅞ inch outer diameter, for a fluid that may not require as much exposure to the light emitted from the lamps 152. Thus, an operator may pass a fluid, such as the solution or a biological fluid like blood, through the photo reactor 100 with tubing 132 having a first outer diameter, such as ¼ inch. Next the operator may remove the tubing 132 from the photo reactor 100 and replace it with tubing 132 having a larger diameter, such as ⅞ inch and passing another fluid through the photo reactor 100 different from first fluid. Because the inner and outer shields 134, 136 do not contact the fluid under normal operating conditions, they are configured to remain in the photo reactor 100 during and/or after replacement of the tubing 132.

The photo reactor 100 may include a fan (not shown) housed in the space formed by the base 102 to help force air upward along the tubing 132, inner and outer shields 134, 136, and the lamp subassembly 150. In other embodiments an additional or alternative fan may be placed in the space formed by the top cap 112 to help force air out of the photo reactor 100 or downwardly through the photo reactor 100 along the aforementioned components. Alternatively, a cooling source, such as an air conditioning unit, may be configured to connect to the photo reactor 100 at the upper or lower apertures 106, 116 to force conditioned air along the aforementioned components. The photo reactor 100 may also house ballasts for the fluorescent lamps or such ballasts may be housed externally and wired to the sockets 158. It is foreseen that the sockets 158 may be configured to connected to a ballast or to bypass it for when a non-fluorescent light source is used in the photo reactor 100.

FIGS. 7-10 illustrate another exemplary coil subassembly 130a. The coil subassembly 130a includes an inner core 134a (shown in FIG. 10) defining an inner radial portion of a helically wound channel 132a and an outer cylindrical sleeve 136a (shown in FIG. 9) defining an outer radial portion of the helically wound channel 132a. The inner core 134a and the outer cylindrical sleeve 136a may comprise a material configured to at least be partially translucent to UV light, such as cyclic olefin copolymer or cyclic olefin polymer and may be formed by injection molding. The inner core 134a and the outer cylindrical sleeve 136a may be solvent bonded or ultrasonically welded to one another, as illustrated in FIG. 8, when the respective radial portions of the helically wound channel 132a are aligned. The coil subassembly 132a may be interchangeable in form and function with the coil assembly 130, including the tubing 132, inner cylindrical shield 134, outer cylindrical shield 136, top vent 138, and bottom vent 140. In some embodiments, the coil subassembly 130a may have channels 132a with a rectangular cross-section. Further, in some embodiments, the inner core 134a and outer sleeve 136a may be rectangular, which may permit the inner and/or outer lamps 152 to be arranged in linear along the coil subassembly 132a.

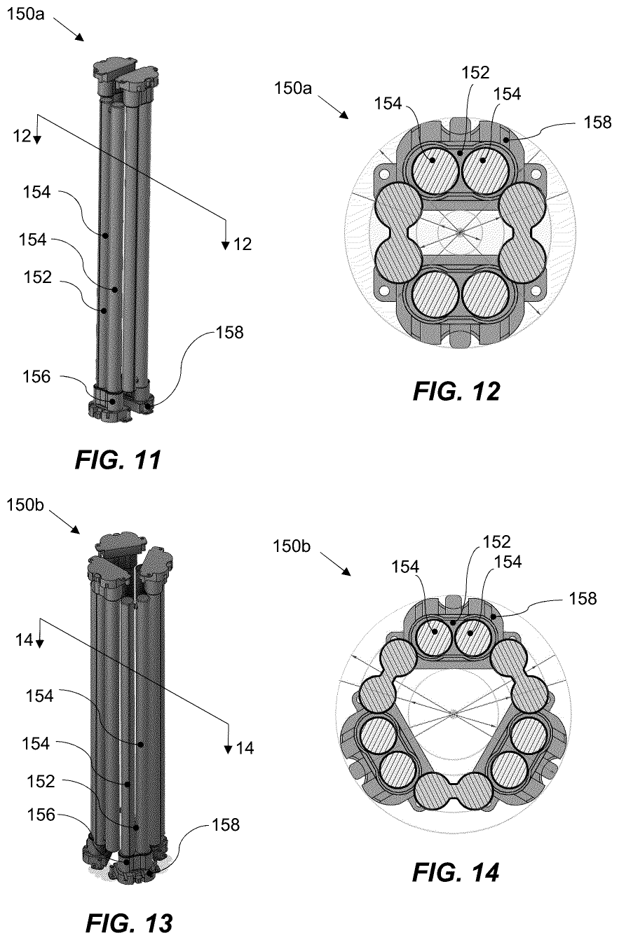

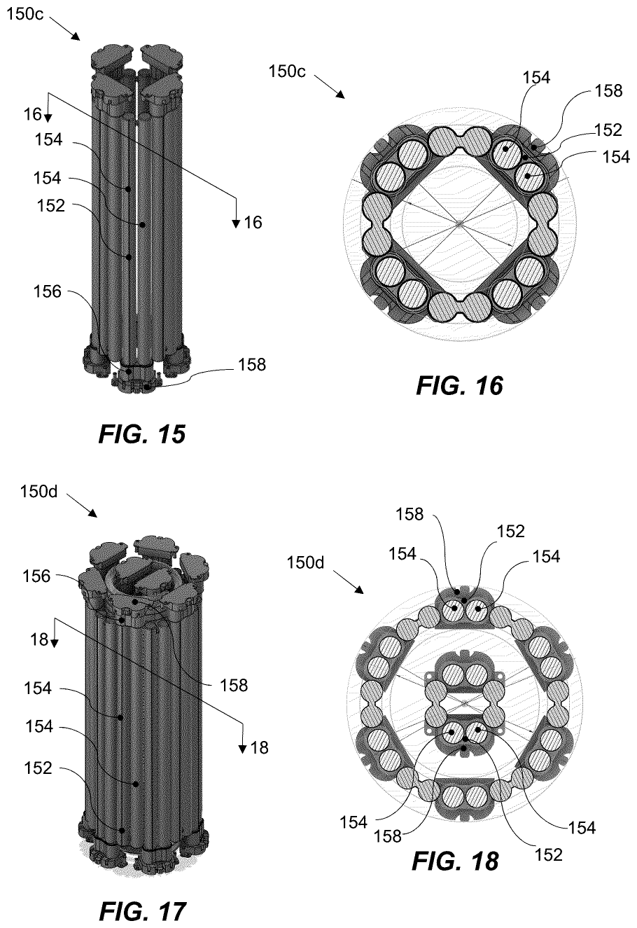

FIGS. 11 and 12 illustrate another lamp subassembly 150a similar to the lamp subassembly 150 but with only inner lamps 152. FIGS. 13 and 14 illustrate another lamp subassembly 150b similar to the lamp subassembly 150 but with six lamps 152 which may be configured as inner or outer lamps 152. FIGS. 15 and 16 illustrate another lamp subassembly 150c similar to the lamp subassembly 150 but with eight lamps 152 which may also be configured as inner or outer lamps 152. FIGS. 17 and 18 illustrate another lamp subassembly 150d similar to the lamp subassembly 150 but with six outer lamps 152. Each of lamp subassemblies 150a-150d may be interchangeable with lamp subassembly 150.

In another embodiment, the method is applied in a flow-through bioreactor such as a Couette flow device (not shown). The Couette flow device may comprise a transparent shell and LED lights surrounding the transparent shell. In some embodiments, an inner cylinder may include a thin optical shell on the outer circumference. A rotating inner cylinder may provide convection for the optical reacting layer. The inner cylinder may rotate at a sufficient speed to induce Rayleigh-Taylor vortices for efficient mixing of the mixture in the outer shell.

The inner cylinder may be suspended within an outer cylinder. The inner cylinder may be positioned between opposing ring magnets to help keep the inner cylinder centered and also to help control the axial position. The ring magnets may be radially polarized. This means that the north poles are on the outside and the south poles are on the inside, or vice versa. The rings on the stationary outer cylinder and the rotating inner cylinder may be offset axially. Either both rings on the outer cylinder are outside or inside the rings on the inner cylinder.

The inner cylinder may be constructed of thin wall aluminum. This may allow creation of eddy-currents to control the spin of the inner cylinder. Iron features may be bonded to the cylinder to create a salient-pole motor, but too much iron may create a tendency to pull the cylinder to the side wall and would have to be balanced against ring magnet force.

The size of the inner and outer cylinders may be set to provide the proper annular spacing. If the spacing is too small turbulent flow will not occur. If the spacing is too large, light penetration may be compromised.

Rotation of the inner cylinder may be controlled by a set of multiphase windings on the outer cylinder. Nominally this may be considered a three phase system. The rotating phases may drag the inner cylinder in rotation by the creation of eddy currents. A variable frequency drive should be used to allow variation of rotational speed. Light sources as discussed above may be used for illumination. Flexible OLED sheets may also be used for illumination. In addition, it may be possible to implement additional lights in the center of the stationary cylinder or on the outer surface of the rotating cylinder; these would need to be powered by inductive coupling.

The flowrate of the fluids can be controlled by the speed of the pumps. A main pump may be used to control the overall flow rate and the riboflavin pump may be slaved to the main pump to maintain the proper RF/liquid ratio.

The spinning inner cylinder may be removed and heat sterilized, by boiling and/or bleach.

In another embodiment, the method is applied to sterilize surfaces. Any type of surface could be sterilized with this method, including hard surfaces and soft surfaces. The surface may be porous or non-porous and may comprise fabric, metal, ceramic, porcelain, plastic, stone, artificial stone, cement, vinyl, etc. In some embodiments, the surface is one or more surfaces of medical equipment. For example, surgical masks, hospital equipment, first responder equipment, etc.

By way of a non-limiting example, a surface is sprayed with photosensitizer (e.g., riboflavin)-infused solution and then activating the deposited solution with light (primarily in the blue/UV spectrum) to activate the photosensitizer (e.g., riboflavin) and increase the efficacy of disinfection. The photosensitizer (e.g., riboflavin)-infused solution may comprise water. In some embodiments, the photosensitizer (e.g., riboflavin)-infused solution is photosensitizer (e.g., riboflavin)-water.

In some embodiments, a deposition device deposits photosensitizer (e.g., riboflavin) to the surface to be sterilized and then photosensitizer (e.g., riboflavin) is activated with light prior to the surface leaving the deposition device. In other embodiments, the surface is exposed to light after leaving the deposition device.

In some embodiments, the sprayer is separate from the light source. In other embodiments, the sprayer and light source are integrated into the same device/apparatus. In some embodiments, the light source is integrated into the deposition device. For example, a spray nozzle may have the light source integrated into the nozzle. The deposition device may be a spray device but could also include other forms of deposition, including but not limited to: stream, bulk liquid (i.e. bucket sloshing), stream, roller, fogger, etc.

In some embodiments, the light source is nominally presumed to be a series (which could be as few as one) of LED devices emitting in the UV and blue range. The light source could also be a broad range source (i.e. incandescent source, fluorescent source, arc source, metal halide source, etc.). The light source can be a single light unit (single LED, single incandescent, etc.) or a multiplicity of units.

As discussed above, a certain amount of energy is required to activate the photosensitizer (e.g., riboflavin) in a given volume of water/photosensitizer (e.g., riboflavin) solution. In an embodiment, the method comprises an embodiment which delivers water and light at the appropriate rate. More specifically, the ratio of water molecules and photons delivered would remain proportional. While this ratio is desirable for energy efficiency (important to match the electrical supply with the water supply in an implementation unit), it may not be required.

One way to maintain an approximate water/light ratio is to maintain approximately similar dispersion angles for both the water supply and the light supply. It is noted that the dispersion angle isn't strictly the spray angle. If an annular water spray is used, or an annular light ring is used—or if the water sprays and light sources are intermixed—the water spray angle and light dispersion angles could be somewhat different.

In some embodiments, the solution being sprayed may cool the light source, such as an LED. The methods disclosed herein, can also be adapted for small scale and large scale sanitization/viral particle inactivation. By way of non-limiting examples, the present disclosures can be used for a backpack sprayer or an automated hand sanitizer.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A device for emitting photons into a fluid, the device comprising:
a fluid channel configured to transfer the fluid from an inlet to an outlet thereof and the fluid channel having a helical pathway wound about a longitudinal axis;
at least one inner light source adjacent the fluid channel, the at least one inner light source positioned between the fluid channel and the longitudinal axis; and
at least one outer light source adjacent the fluid channel, the fluid channel positioned between the outer light source and the longitudinal axis.

2. The device of claim 1, wherein at least one of the inner and outer light sources is mounted at a top surface and extends downwardly parallel with the longitudinal axis and at least a second one of the inner and outer light sources is mounted at a bottom surface axially spaced apart from the top surface and extends upwardly parallel with the longitudinal axis.

3. The device of claim 1, wherein the at least one outer light source includes a plurality of light sources arranged circumferentially around the helical pathway.

4. The device of claim 1, wherein at least one of the inner and outer light sources is a fluorescent light source.

5. The device of claim 4, wherein at least a second one of the inner and outer light sources is an LED light source.

6. The device of claim 4, wherein at least one of the inner and outer light sources is a narrowband wavelength light source.

7. The device of claim 4, wherein at least two of the inner and outer light sources have different peak wavelengths.

8. A device for emitting photons into a fluid, the device comprising:
a fluid channel configured to transfer the fluid from an inlet to an outlet thereof; and
at least one first light source and at least one second light source adjacent the fluid channel, the at least one first light source having a different peak wavelength than the at least one second light source, and the at least one first light source configured to have a narrowband wavelength output;
wherein at least one of the first and second light sources is a fluorescent light source.

9. The device of claim 8, wherein at least one of the first and second light sources is an LED light source.

10. The device of claim 8, wherein the at least one first light source and the at least one second light source are florescent light sources.

11. The device of claim 8, wherein at least one of the first and second light sources has a peak UV-B wavelength.

12. The device of claim 8, wherein at least one of the first and second light sources has a peak UV-C wavelength.

13. The device of claim 12, wherein at least one of the first and second light sources has a peak UV-C wavelength.

14. The device of claim 8, wherein at least one of the first and second light sources has a peak wavelength outside of UV-B and UV-C.

15. A method of directing photons into fluid along a pathway between an inlet and an outlet, the method comprising:
emitting, with at least one first light source, photons having a narrowband first peak wavelength into the fluid pathway; and
emitting, with at least one second light source, photons having a second peak wavelength different than the first wavelength into the fluid pathway;
wherein at least one of the first and second light sources is a fluorescent light source.

16. The device of claim 15, wherein at least one of the first and second light sources is an LED light source.

17. The device of claim 15, wherein the at least one first light source and the at least one second light source are florescent light sources.

18. The device of claim 15, wherein at least one of the first and second light sources has a peak UV-B wavelength.

19. The device of claim 15, wherein at least one of the first and second light sources has a peak UV-C wavelength.

20. The device of claim 19, wherein at least one of the first and second light sources has a peak UV-C wavelength.

21. The device of claim 15, wherein at least one of the first and second light sources has a peak wavelength outside of UV-B and UV-C.
